(19) United States
(12) Patent Application Publication
(10) Pub. No.: US 2017/0223484 A1
(43) Pub. Date: Aug. 3, 2017

(54) SKIN MOISTURE TESTING DEVICE

(71) Applicant: Shenzhen Hali-Power Industrial Co., Ltd., Shenzhen (CN)

(72) Inventor: XIAOMIN ZHU, Chenzhen (CN)

(21) Appl. No.: 15/455,069

(22) Filed: Mar. 9, 2017

Related U.S. Application Data
(63) Continuation-in-part of application No. 15/014,041, filed on Feb. 3, 2016.
Application No. 15/014,041 is a continuation-in-part of application No. 15/013,943, filed on Feb. 2, 2016, now abandoned.
Application No. 15/013,943 is a continuation-in-part of application No. 15/159,768, filed on May 19, 2016.

(51) Int. Cl. H04W 4/00; A61B 5/00

(52) U.S. Cl. CPC H04W 4/008; H04W 4/003; A61B 5/443; A61B 5/002

(57) ABSTRACT

A skin moisture testing system comprises a PCB, a skin moisture testing system arranged on the PCB and including a Bluetooth MCU, a Bluetooth antenna impedance matching unit, a RC integral and diode rectifier unit, a sinusoidal signal unit, a first contact, a second contact, a first spring arranged around the first contact, a second spring arranged around the second contact, and a case. The case includes a housing, an upper cover, and a connecting cover. The PCB is received in the housing. The first contact and the second contact are integrally fanned with the upper cover, and the connecting cover defines a receiving space for receiving the upper cover.

### CROSS REFERENCE TO RELATED APPLICATION

This application is a continuation in part and claims benefit of the following patent properties: (1) U.S. patent application Ser. No. 15/014,041, entitled “SKIN MOISTURE TESTING SYSTEM AND METHOD” filed on Feb. 3, 2016, (2) U.S. patent application Ser. No. 15/013,943, entitled “ULTRAVIOLET DETECTION SYSTEM AND METHOD” filed on Feb. 2, 2016, and (3) U.S. patent application Ser. No. 15/159,768, entitled “SKIN MOISTURE TESTING SYSTEM AND METHOD” filed on May 19, 2016. The above listed applications are hereby incorporated by reference herein as if set forth in its entirety.

### BACKGROUND

1. Technical Field

The present disclosure generally relates to a testing region, and especially relates to a skin moisture testing device.

2. Description of Related Art

Traditional cosmetics can only provide skin moisture; however, the traditional cosmetics do not know about skin moisture conditions, and cannot provide a scientific and reasonable moisturizing solution to the skin. The present invention aims to let consumers intuitively know his/her own skin condition, and provide the skin moisture in time. We can learn more about our skin, and care for our skin more scientifically and reasonably.

Therefore, a need exists in the industry to overcome the described problems.

### SUMMARY

The disclosure is to offer a skin moisture testing device.

A skin moisture testing device includes a PCB, a skin moisture testing system arranged on the PCB, a first contact, a second contact a first spring arranged around the first contact and comprising a first end connected to the PCB, a second spring arranged around the second contact and comprising a second end connected to the PCB, and a case. Me skin moisture testing system includes a Bluetooth MCU, a Bluetooth antenna impedance matching unit including, an IC matching circuit and a terminal matching circuit, a RC integral and diode rectifier unit, and a sinusoidal signal unit. The case includes a housing, an upper cover, and a connecting cover, the PCB received in the housing, the first contact and the second contact integrally formed with the upper cover; the connecting cover defining a receiving space for receiving the upper cover, the housing comprising an accommodating room located at an end thereof, a first hole for receiving the first contact located at a bottom face of the accommodating room, a second hole for receiving the second contact located at the bottom face of the accommodating room, and a plurality of first projections located in an inner sidewall of the accommodating room, the connecting cover comprising an inserting portion received in accommodating room and a plurality of first receiving grooves located at a sidewall of the inserting portion to receive the first projections. The sinusoidal signal unit transmits a square wave high frequency sinusoidal signal emitted by the Bluetooth MCU to the first contact, after a current passing through the skin, the current being fed to the Bluetooth MCU through the second contact, and the sinusoidal signal changed to a first signal, the first signal filtered and shaped by the RC integral and diode rectifier unit to a second signal, the Bluetooth MCU sampling the second signal to get a plurality of samples, the plurality of samples quantified to get a semaphore value. Signals indicative of the semaphore values are transmitted to an application (APP) on a terminal by the connection between the Bluetooth antenna impedance matching unit to the terminal, and the APP compares the semaphore value with a database in the terminal to obtain a skin moisture content value. The Bluetooth antenna impedance matching unit matches with the Bluetooth MCU, and partly matches with an on board antenna of the Bluetooth antenna.

Wherein, the connecting cover comprises a plurality of second projections protruding from a bottom face of the inserting portion, the housing defines a plurality of second receiving moves located the bottom face of the accommodating room to receives the second projections.

Wherein the housing comprises a plurality of third projections located at the inner sidewall of accommodating room, the connecting cover defines a plurality of depressions located at the sidewall of the inserting portion.

Wherein each of the third projections has a shovel shape, and each of the depressions has a shovel shape and comprises a first depression and a second depression, wherein a depth of the first depression is greater than that of the second depression.

Wherein the connecting cover comprises a plurality of ribs projecting from the sidewall of the inserting portion, and the housing defines a plurality of slots to receive the ribs.

Wherein the skin moisture testing device comprises a cap covering the connecting cover and comprising a pair of first fixing portions each having a B shape, the connecting cover comprises a pair of second fixing portions engaging with the first fixing portions to mount the cap to the connecting cover.

Wherein each of the first fixing portions comprises a first protruding portion, a second protruding portion, and a slot between the first protruding portion and the second protruding portion, wherein a height of the second protruding portion is less than that of the first protruding portion.

Wherein each of the second fixing portions comprises a first receiving, aperture a second receiving aperture, and a post between the first receiving aperture and the receiving aperture, wherein the first protruding portion is received in the first receiving aperture, the second protruding portion is received in the second receiving aperture, and the post is received in the slot.

Wherein a depth of the second receiving aperture is slightly less than that of the first receiving aperture.

Wherein the first protruding portion has a first cambered face, and the second fixing portions comprises a second cambered face located at a bottom face of the first receiving aperture to engage with the first cambered face to facilitate the first fixing portion mounted to the second fixing portion.

Compared to the traditional skin moisture testing device, the skin moisture testing device of the present invention is motivated by a pulse signal, the skin moisture testing method can effectively and accurately test skin moisture value. The skin moisture testing method also matches, with a lower powered Bluetooth MCU to test user's skin for a long time, then the tested skin moisture value can be visually displayed on the APP of the terminal device, the testing data can be made to form a graph, such that the skin moisture testing method can test comprehensive skin nutrition moisture percentage under the basis of an guarantee of no dais aging to the skin, and the skin moisture testing method can guide the care of the skin.

### BRIEF DESCRIPTION OF THE DRAWINGS

Many aspects of the present embodiments can be better understood with reference to the following drawings. The components in the drawings are not necessarily drawn to scale, the emphasis instead being placed upon clearly illustrating the principles of the present embodiments. Moreover, in the drawings, all the views are schematic, and like reference numerals designate corresponding parts throughout the several views.

FIG. 1 is a schematic diagram of a skin moisture testing system;

FIG. 2 is an isometric view of a circuit structure;

FIG. 3 is an isometric view of a Bluetooth MCU;

FIG. 4 is an isometric view of a Bluetooth antenna impedance matching unit;

FIG. 5 is a structure diagram of a sinusoidal signal unit;

FIG. 6 is a structure diagram of a RC integral and diode rectifier unit;

FIG. 7 is a flow chart of a method of testing a skin moisture value based on the skin moisture testing system;

FIG. 8 is an exploded perspective view of a skin moisture testing device of the present invention;

FIG. 9 is a partly assembled view of a housing of the skin moisture testing device of FIG. 8;

FIG. 10 is an assembled view of a PCB, a pair of springs, and a pair of contacts of the skin moisture testing device of FIG. 8;

FIG. 11 is a perspective view of a cap of the skin moisture testing device of FIG. 8;

FIG. 12 is a perspective view of a connecting cover of the skin moisture testing device of FIG. 8; and

FIG. 13 is a perspective view of a housing of the skin moisture testing device of FIG. 8.

### DETAILED DESCRIPTION

The disclosure is illustrated by way of example and not by way of limitation in the figures of the accompanying drawings, in which like reference numerals indicate similar elements. It should be noted that references to “an” or “one” embodiment in this disclosure are not necessarily to the same embodiment, and such references can mean “at least one” embodiment.

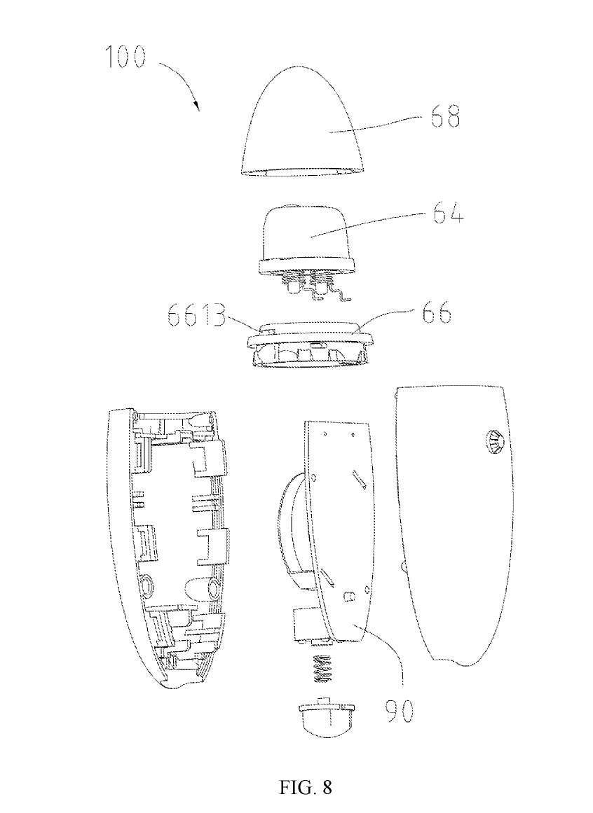

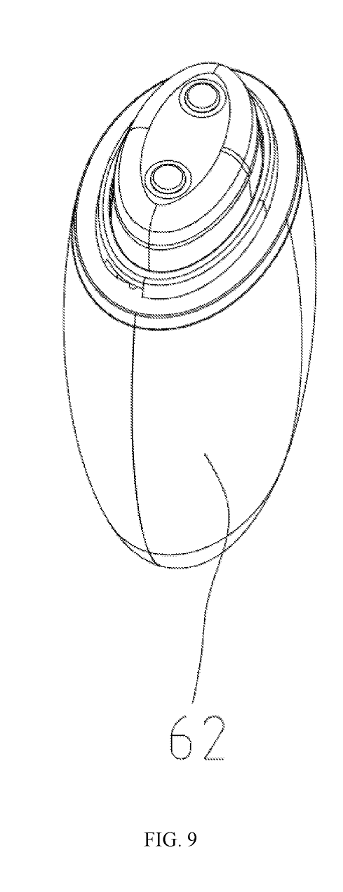

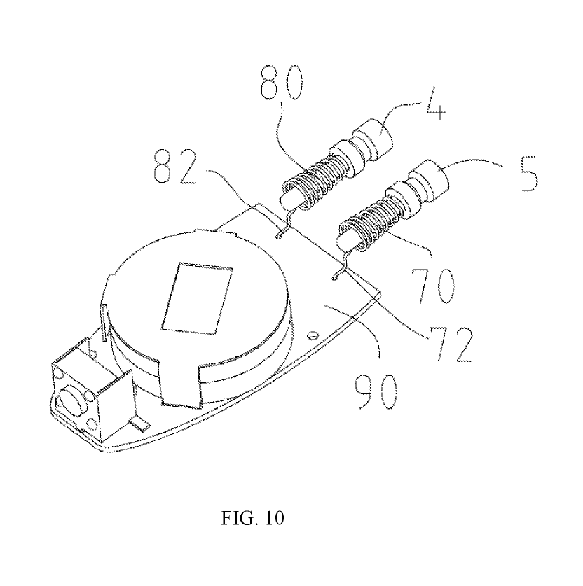

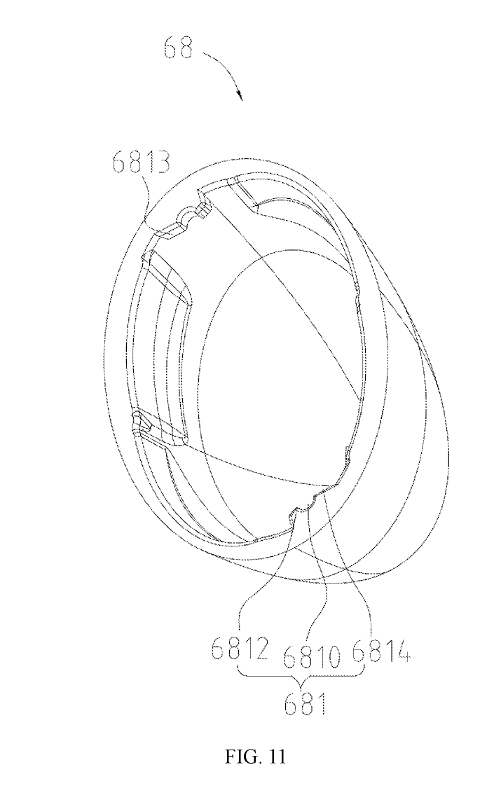

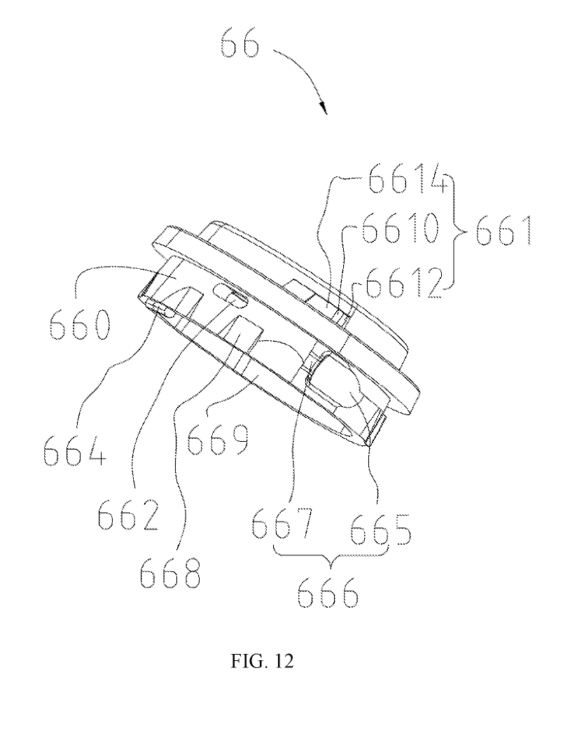

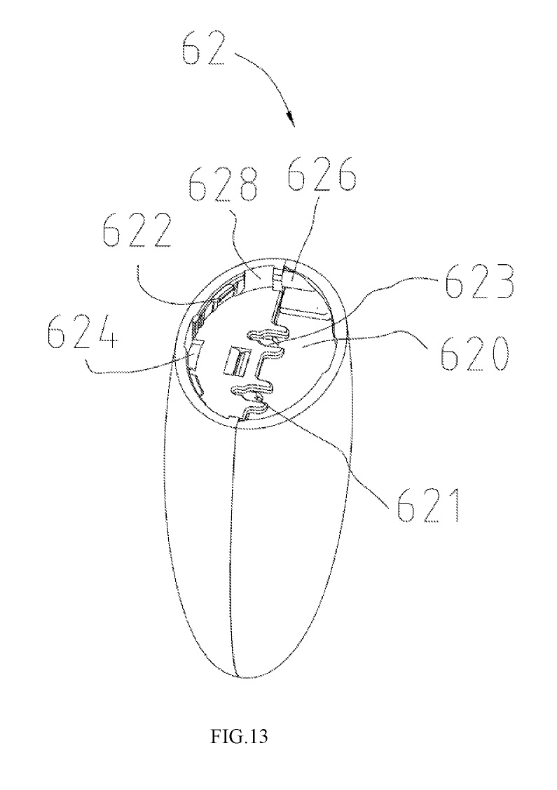

Referring to FIGS. 8-13, the skin moisture testing device 100 includes a PCB 90, a first contact 4, a second contact 5, a first spring 80 arranged around the first contact 4 and including a first end 82 connected to the PCB 90, a second spring 70 arranged around the second contact 5 and including a second end 72 connected to the PCB 90, a case including a housing 62, an upper cover 64, and a connecting cover 66. The PCB 90 is received in the housing 62. The first contact 4 and the second contact 5 are integrally formed with the upper cover 64. The connecting cover 66 defines a receiving space 669 for receiving the upper cover 64. The housing 62 includes an accommodating room 620 located at an end thereof; a first hole 621 for receiving the first contact 4 and located at a bottom face of the accommodating room 620, a second hole 623 for receiving the second contact 5 and located at the bottom face of the accommodating room 620, and a plurality of first projections 622 located in an inner sidewall of the accommodating room 620. The connecting cover 66 includes an inserting portion 660 received in accommodating room 620 and a plurality of first receiving grooves 662 located at a sidewall of the inserting portion 660 to receive the first projections 622.

The connecting cover 66 includes a plurality of second projections 664 protruding from a bottom face of the inserting portion 660, and the housing 62 defines a plurality of second receiving grooves 624 located the bottom face of the accommodating room 620 to receive the second projections 664.

The housing 62 includes a plurality of third projections 26 located at the inner sidewall of accommodating room 620, and the connecting cover 66 defines a plurality of depressions 666 located at the sidewall of the inserting portion 660.

In one embodiment, each of the third projections 626 has a shovel shape, and each of the depressions 666 has a shovel shape and comprises a first depression 665 and a second depression 667, wherein a depth of the first depression 665 is greater than that of the second depression 667.

The connecting cover 66 includes a plurality of ribs 668 projecting from the sidewall of the inserting portion 660, and the housing 62 defines a plurality of slots 628 to receive the ribs 668.

In one embodiment, each of the ribs 668 has a different thickness along an axial direction of the inserting portion 660 to locate the connecting cover 66 in the housing 62.

The skin moisture testing device includes a cap 68 covering the connecting cover 66 and including a pair of first fixing portions 681 each having B shape. The connecting cover 66 includes a pair of second fixing portions 661 engaging with the first fixing portions 681 to mount the cap 68 to the connecting cover 66.

In one embodiment, each of the first fixing portions 681 includes a first protruding portion 6812, a second protruding portion 6814, and a slot 6810 between the first protruding portion 6812 and the second protruding portion 6814, wherein a height of the second protruding portion 6814 is less than that of the first protruding portion 6812.

Each of the second fixing portions 661 includes a first receiving aperture 6612, a second receiving aperture 6614, and a post 6610 between the first receiving aperture 6612 and the second receiving aperture 6614. Wherein the first protruding portion 6812 is received in the first receiving aperture 6612, the second protruding portion 6814 is received in the second receiving aperture 6614, and the post 6610 is received in the slot 6810.

In one embodiment, a depth of the second receiving aperture 6614 is slightly less than that of the first receiving aperture 6612.

The second protruding portion 6814 has a first cambered face 6813, and the second fixing portions 661 comprises a second cambered face 6613 located at a bottom face of the second receiving aperture 6614 to engage with the first cambered face 6813 to facilitate the first fixing portion 681 mounted to the second fixing portion 661.

The skin moisture testing device 100 includes a skin moisture testing system arranged on the PCB 90 and including a Bluetooth MCU (Micro Control Unit) 1, a Bluetooth antenna impedance matching unit 2, an A/D (Analog/Digital) detecting unit 3 which can also be defined as a RC integral and diode rectifier unit which includes a RC integral circuit 31 and a diode rectification 33, a sinusoidal signal unit 17, wherein the first contact 4 is presented as a Skin 1, and the second contact 5 is presented as a Skin 2, wherein the resistance of the user's body constitutes the resistor (R), and the capacitor C13 and the above resistor cooperatively constitute the RC integral portion of the RC integral and diode rectifier unit, and the RC integral and diode rectifier unit is configured to process the alternating current (AC) signals. The Bluetooth MCU 1 has the following functions, such as a A/D (Analog/Digital) testing 11, a LED indication controlling 13 (light emitting diode, LED), an antenna impedance matching 19, the Bluetooth MCU 1 also has a power button 15, the antenna impedance matching unit 2 includes an IC matching circuit 21 and a terminal matching circuit 23, the sinusoidal signal unit 17 can transmit the sinusoidal signal which can be a square wave of a high frequency sinusoidal the sinusoidal signal is emitted by the Bluetooth MCU 1 and transmitted to the first contact 4, after a current passes through the skin, the current is fed to the Bluetooth MCU 1 through the second contact 5, and the sinusoidal signal is changed to a first signal, the first signal can be filtered and shaped by the A/D detecting unit 3 to a second signal, the Bluetooth MCU 1 samples the second signal to get a plurality of samples, and then the samples are quantified to get a semaphore value. The signals indicative of the semaphore values are transmitted to an application (APP) on the terminal 50 by the connection between the Bluetooth antenna impedance matching unit 2 to the terminal 50. The application (APP) compares the semaphore value with a database in the terminal 50 to obtain a skin moisture content value. The Bluetooth antenna impedance matching unit 2 is responsible for matching with the Bluetooth MCU 1, and partly matching with an on board antenna, such that the skin moisture testing system of the present invention can remotely and reliably transport the data.

The high frequency signal can be 4 KHz±500 Hz sinusoidal signal, such as 3700 Hz, 3800 Hz, 3900 Hz, 4000 Hz, 4300 Hz, and 4400 Hz.

A ninth terminal of the Bluetooth MCU 1 is connected with the RC integral and diode rectifier unit, and can be used to test integral values.

A fifth terminal of the Bluetooth MCU 1 is connected with a resistor R8 and a light emitting diode (LED), and can be used to control a state of the light emitting diode.

A fifteenth terminal of the Bluetooth MCU emits 4 KHz±500 Hz sinusoidal signal and transmits the 4 KHz±500 Hz sinusoidal signal to the first contact 4.

A thirty first terminal of the Bluetooth MCU 1 and a thirty second terminal of the Bluetooth MCU 1 are connected with the Bluetooth antenna impedance matching unit 2.

A circuit of the Bluetooth antenna impedance matching unit 2 includes a first coil L1, a second coil L2, a third coil L3, a fourth coil L4, a third capacitor C3, a fourth capacitor C4, a fifth capacitor C5, a sixth capacitor C6, a fourteenth capacitor C14, a sixteenth capacitor C16, and an antenna; the antenna, the sixteenth capacitor C16 the fourteenth capacitor C14, the fifth capacitor C5, the first coil L1, the second coil L2, the third capacitor C3 are cascaded with each other in sequence, and then grounded, a lead between the first coil L1 and the fifth capacitor C5 is connected with the thirty second terminal of the. Bluetooth MCU 1, another lead between the first coil L1 and the second coil L2 is connected with the thirty first terminal of the Bluetooth MCU 1, a lead between the second coil L2 and the third capacitor C3 is connected with a thirtieth terminal of the Bluetooth MCU 1, the fourth capacitor C4 is connected with the fifth capacitor C5 and the fourteenth capacitor C14, and then grounded, the fourth capacitor C4 is located between the fifth capacitor C5 and the fourteenth capacitor C14, the fourteenth capacitor C14 and the sixteenth capacitor Cl6 are connected with the sixth capacitor C6 and the second coil L3 in parallel, and then grounded, the fourth coil L4 is connected with the sixteenth capacitor C16 and the antenna, and then grounded, the fourth coil L4 is located between the sixteenth capacitor C16 and the antenna.

In at least one exemplary embodiment, the Bluetooth MCU 1 can be a chip which can have forty eight terminals, a first terminal of the chip is connected with a power VCC 6 (Volt. Current Condenser, VCC), and the first terminal of the chip is grounded through a seventh capacitor C7; a seventh terminal of the chip is connected with a switch (not shown); a twelfth terminal of the chip is connected with the power VCC 6, and the twelfth terminal of the chip is grounded through an eleven capacitor C11; a thirteenth terminal of the chip is grounded; a twenty fourth terminal is grounded by a first resistor R1, a twenty third terminal is connected with a D point, the twenty fourth terminal is connected with a C point, a G point is grounded, and the D point, the C point and the G point are burning points; a twenty ninth terminal is grounded through a tenth capacitor C10, a thirty third terminal and a thirty fourth terminal are both grounded, a thirty fifth terminal and a thirty sixth terminal are both connected with the power VCC 6, the thirty fifth terminal and the thirty sixth terminal are grounded through a ninth capacitor C9, a thirty seventh terminal is grounded through a first capacitor C1, a thirty eighth terminal is grounded through the second capacitor C2, a crystal oscillator Y1 is located between the thirty seventh terminal and the thirty eighth terminal, and the crystal oscillator Y1 is connected with the thirty seventh terminal and the thirty eighth terminal, a thirty ninth terminal is grounded through an eighth capacitor C8.

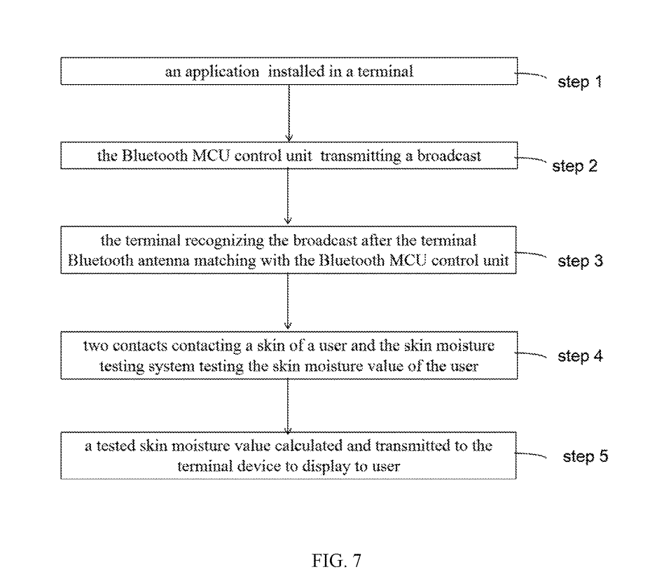

Referring to FIG. 7, FIG. 7 is a flow chart, of a method of testing a skin moisture value based on the skin moisture testing system. The method of testing the skin moisture value based on the skin moisture testing system, the method comprising: step 1: an application 51 installed in an terminal 50 having a Bluetooth antenna 53, the terminal 50 being a mobile phone or a tablet personal computer; step 2: the Bluetooth MCU 1 transmitting a broadcast by pressing power button 15; step 3: the terminal device 5 recognizing the broadcast after the terminal Bluetooth antenna 53 matching with the Bluetooth MCU 1; step 4: the two contacts 4, 5 contacting a skin of a user and the skin moisture testing system testing the skin moisture value of the user; and step 5: a tested skin moisture value calculated by the Bluetooth MCU 1 and transmitted to the terminal device 5 to display to the user.

It is to be understood that, when the Bluetooth MCU 1 sends out the broadcast, the user can select to boot the Bluetooth antenna 53 on the terminal 50, then the user can select a corresponding service set identifier (SSID) of the broadcast on an APP interface of the terminal 50 after the terminal 50 are matched and connected with the skin moisture testing system, two contacts 4, 5 can be contacted with the skin.

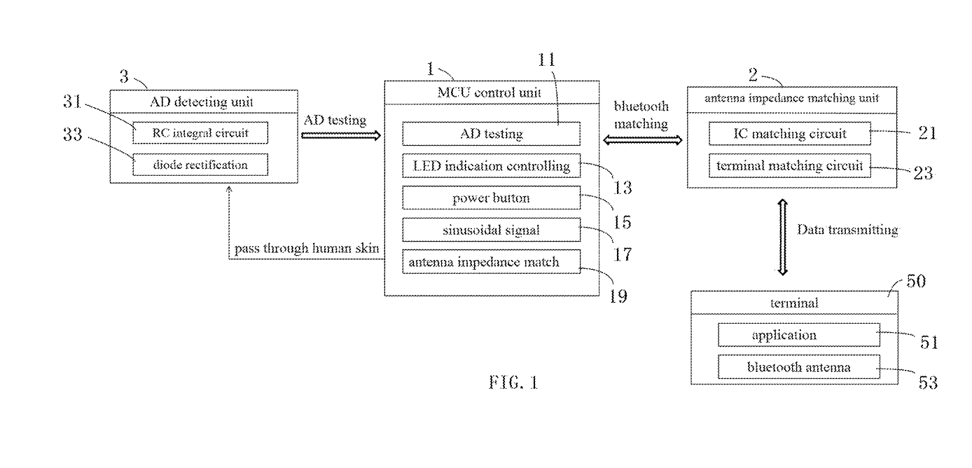

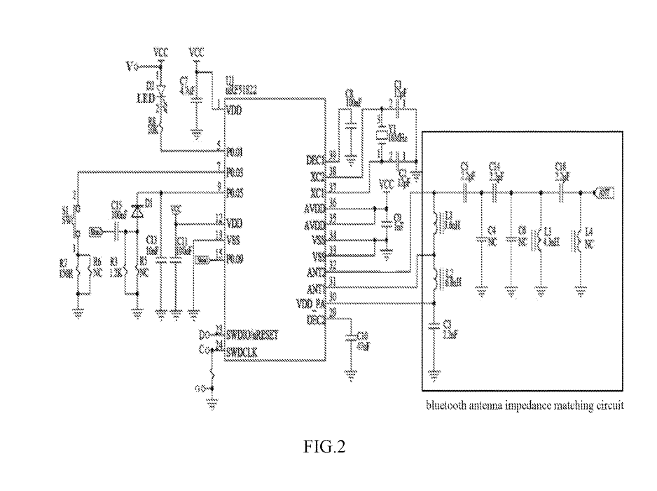

Referring to FIG. 1, FIG. 1 is a schematic diagram of the skin moisture testing system, the skin moisture testing system of the present invention includes the Bluetooth MCU 1, the Bluetooth antenna impedance matching unit 2, the RC integral and diode rectifier unit which can also be defined as the A/D detecting unit 3 and the sinusoidal signal unit 17.

The skin moisture testing system can be connected with the terminal 50 and send data to the terminal 50 through the Bluetooth. The terminal 50 can be a phone, a tablet personal computer and so on. The skin moisture testing system can test percentage of comprehensive nutrient water in the skin based on an assurance of not damaging the skin, and a skin care can be guided by a skin moisture testing product using the skin moisture testing system. Testing parts of the skin can include head, face, eye socket, neck, and so on. Firstly, the application 51 can be installed on the terminal 50, and the application 51 can be a Mili Pure application. The Bluetooth MCU 1 transmits a broadcast, and the terminal device recognizes the broadcast after the terminal Bluetooth snatching, with the Bluetooth MCU 1. The two contacts 4, 5 contact the skin of a user and the skin moisture testing system tests the skin moisture value of the user. A tested skin moisture value is calculated by the Bluetooth MCU 1 and is transmitted to the terminal 50 to display to the user.

The square wave of the high frequency sinusoidal signal can be applied on the first contact 4 which is contacted with the skin, the high frequency sinusoidal signal can be 4 KHz±500 Hz sinusoidal signal, a weak small current passes through the skin and then return back through the second contact 5, after the sinusoidal signal is signal filtered, and shaping treated, the Bluetooth MCU 1 starts sampling to get a plurality of samples, the samples are quantified to get a semaphore value, the semaphore value can be transformed into a corresponding skin impedance value, a transformation equation can be established through a data fitting analysis, finally the skin impedance value is translate into skin moisture content value corresponding to the skin impedance value. Tested skin moisture value can be calculated by the Bluetooth MCU algorithm, and then be sent to the application 51 of the terminal 50, such that user can observe the value visually.

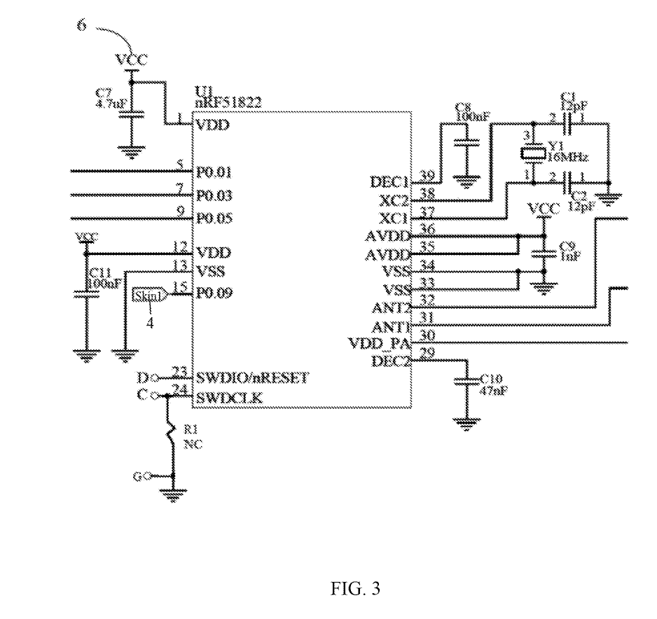

Referring to FIG. 3, FIG. 3 is an isometric view of the Bluetooth MCU 1; the Bluetooth MCU 1 is responsible for A/D sampling testing, sending and receiving the data signal, logic control signal, and sending high frequency sinusoidal signal The ninth terminal is connected with the testing integral value. The fifth terminal is connected with the resistor R8 and the light emitting diode to control a state of the light emitting diode. The fifteenth terminal sends out 4 KHz sinusoidal signal to the first contract 4. The thirty first terminal and the thirty second terminal are connected with the Bluetooth antenna impedance matching unit 2.

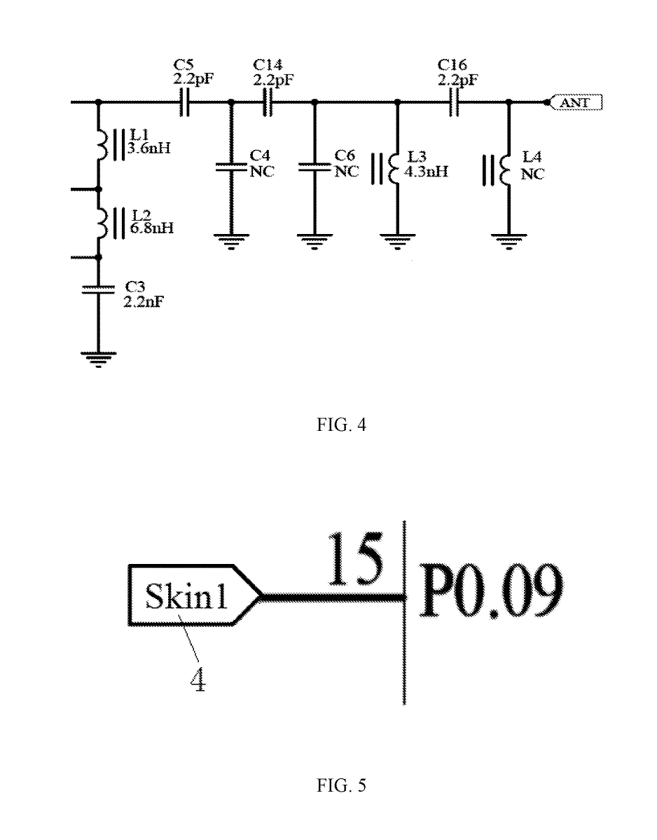

Referring to FIG. 4, FIG. 4 is an isometric view of the Bluetooth antenna impedance matching unit 2, the Bluetooth antenna impedance matching unit 2 is responsible for matching with the Bluetooth MCU 1, and partly matching with the on board antenna, such that the skin is e testing product using the skin moisture testing system can transmit the data to the terminal 50 reliably and remotely.

Referring to FIG. 5, FIG. 5 is a structure diagram of the sinusoidal serial unit, the sinusoidal signal unit 17 is responsible for transmitting 4 KHZ sinusoidal signal emitted by the MCU 1 to the first contact 4.

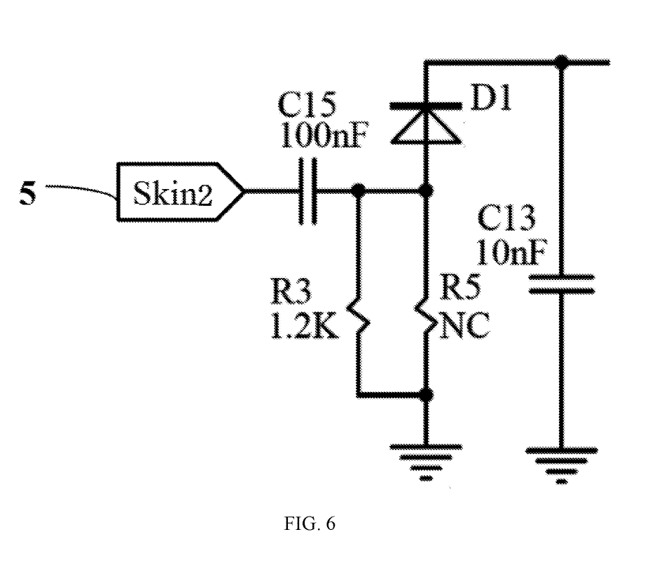

Referring to FIG. 6, FIG. 6 is a structure diagram of the RC integral and diode rectifier unit, a sinusoidal signal emitted by the first contact 4 can pass through the skin, and return to the RC integral and diode rectifier unit through the second contract 5 of the skin moisture testing product using the skin moisture testing system, at this time the sinusoidal signal has already changed, the changed sinusoidal signal is integrated, filtered and shaping treated through the RC circuit 31 which is formed by the fifteenth capacitor C15 and the resistor R8, then the changed sinusoidal signal is returned back to Bluetooth MCU 1, and the Bluetooth MCU starts A/D sampling to get a plurality of samples, the samples are quantified to get a semaphore value, the semaphore value is transformed into corresponding skin impedance value. Then the skin impedance value is sent to the terminal device.

Although the features and elements of the present disclosure are described as embodiments in particular combinations, each feature or element can be used alone or in other various combinations within the principles of the present disclosure to the full extent indicated by the broad general meaning of the terms in which the appended claims are expressed.

What is claimed is:

1. A skin moisture testing device, comprising,
a PCB;
a skin moisture testing system arranged on the PCB and comprising a Bluetooth MCU (Micro Control Unit), a Bluetooth antenna impedance matching unit including an IC matching circuit and a terminal matching circuit, a RC integral and diode rectifier unit, and a sinusoidal signal unit;
a first contact
a second contact;
a first spring arrange around the first contact and comprising a first end connected to the PCB;
a second spring arranged around the second contact and comprising a second end connected to the PCB;
a case comprising a housing, an upper cover, and a connecting cover, the PCB received in the housing, the first contact and the second contact integrally formed with the upper cover, the connecting cover defining a receiving space for receiving the upper cover, the housing comprising an accommodating room located at an end thereof, a first hole for receiving the first contact located at a bottom face of the accommodating room, a second hole for receiving the second contact located at the bottom face of the accommodating room, and a plurality of first projections located in an inner sidewall of the accommodating room, the connecting cover comprising an inserting portion received in accommodating room and a plurality of first receiving grooves located at a sidewall of the inserting portion to receive the first projections;
the sinusoidal signal unit transmitting a square wave high frequency sinusoidal signal emitted by the Bluetooth MCU to the first contact, after a current passing through the skin, the current being fed to the Bluetooth MCU through the second contact, and the sinusoidal signal changed to a first signal, the first signal filtered and shaped by the RC integral and diode rectifier unit to a second signal, the Bluetooth MCU sampling the second signal to get a plurality of samples, the plurality of samples quantified to get a semaphore value;
signals indicative of the semaphore values being transmitted to an application (APP) on a terminal by a connection between the Bluetooth antenna impedance matching unit to the terminal, and the APP comparing the semaphore value with a database in the terminal to obtain a skin moisture content value, the Bluetooth antenna impedance matching unit matching with the Bluetooth MCU, and partly matching with an on board antenna of the Bluetooth antenna.

2. The skin moisture testing device of claim 1, wherein the connecting cover comprises a plurality of second projections protruding from a bottom face of the inserting portion, the housing defines a plurality of second receiving grooves located the bottom face of the accommodating room to receives the second projections.

3. The skin moisture testing device of claim 2, wherein the housing comprises a plurality of third projections located at the inner sidewall of accommodating room, the connecting cover defines a plurality of depressions located at the sidewall of the inserting portion.

4. The skin moisture testing device of claim 3, wherein each of the third projections has a shovel shape, and each of the depressions has a shovel shape and comprises a first depression and a second depression, wherein a depth of the first depression is greater than that of the second depression.

5. The skin moisture testing device of claim 4, wherein the connecting cover comprises a plurality of ribs projecting from the sidewall of the inserting portion, and the housing defines a plurality of slots to receive the ribs.

6. The skin moisture, testing device of claim 1, wherein the skin moisture testing device comprises a cap covering the connecting cover and comprising a pair of first fixing portions each having a B shape, the connecting cover comprises a pair of second fixing portions engaging with the first fixing portions to mount the cap to the connecting cover.

7. The skin moisture testing device of claim 6, wherein each of the first fixing portion comprises a first protruding portion, a second protruding portion, and a slot between the first protruding portion and the second protruding portion, wherein a height of the second protruding portion is less than that of the first protruding portion.

8. The skin moisture testing device of claim 7, wherein each of the second fixing portions comprises a first receiving, aperture a second receiving aperture, and a post between the first receiving aperture and the receiving aperture, wherein the first protruding portion is received in the first receiving aperture, the second protruding portion is received in the second receiving aperture, and the post is received in the slot.

9. The skin moisture testing device of claim 8, wherein a depth the second receiving aperture is slightly less than that of the first receiving aperture.

10. The skin moisture testing device of claim 8, wherein the first protruding portion has a first cambered face, and the second fixing portions comprises a second cambered face located at a bottom face of the first receiving aperture to engage with the first cambered face to facilitate the first fixing portion mounted to the second fixing portion.
